(19) United States
(12) Patent Application Publication
(10) Pub. No.: US 2026/0130919 A1
(43) Pub. Date: May 14, 2026

(54) PRALATREXATE AND COP COMBINATION FOR THE TREATMENT OF PATIENTS WITH PERIPHERAL T CELL LYMPHOMA

(71) Applicant: Acrotech Biopharma Inc., East Windsor, NJ (US)

(72) Inventor: Ashish Anvekar, East Windsor, NJ (US)

(73) Assignee: Acrotech Biopharma Inc., East Windsor, NJ (US)

(21) Appl. No.: 19/440,054

(22) Filed: Jan. 5, 2026

Related U.S. Application Data
(63) Continuation of application No. PCT/US2024/036942, filed on Jul. 5, 2024.
(60) Provisional application No. 63/525,585, filed on Jul. 7, 2023.

(51) Int. Cl. A61K 31/519; A61K 9/00; A61K 31/475; A61K 31/573; A61K 31/675; A61P 35/00

(52) U.S. Cl. CPC A61K 31/519; A61K 9/0019; A61K 31/475; A61K 31/573; A61K 31/675; A61P 35/00

(57) ABSTRACT

The disclosure provides compositions and methods for treating peripheral T-cell lymphoma (PTCL) using a combination of Pralatrexate and COP.

### CROSS REFERENCE TO RELATED APPLICATIONS

The present application is a continuation application of International Patent Application Number PCT/US2024/036942, filed Jul. 5, 2024, which claims priority to and the benefit of U.S. App. No. 63/525,585, filed Jul. 7, 2023, which is hereby incorporated by reference in its entirety.

### BACKGROUND OF THE INVENTION

Lymphoma is a group of blood and lymph tumors that develop from lymphocytes (a type of white blood cell). The term “lymphomas” refers to a variety of disease states, including Non-Hodgkins Lymphoma (NHL); diffuse large B-cell lymphoma (DLBCL); follicular lymphoma (FL); Hodgkin's Disease; Burkitt's Lymphoma; cutaneous T cell lymphoma; primary central nervous system lymphoma, and lymphomatous metastases. In most cases, lymphoma is characterized by the presence of cancerous B cells. However, in peripheral T cell lymphoma (PTCL), the disease state is characterized by the presence of cancerous T lymphocytes.

Treatment approaches for PTCL have mirrored diffuse large B-cell lymphoma, although the majority of PTCLs have an inferior prognosis compared with their B-cell counterparts. Combination chemotherapy with CHOP (Cyclophosphamide, Hydroxydaunorubicin [Doxorubicin], Oncovin [Vincristine], and Prednisone) is the most commonly used first-line treatment and is considered the “standard therapy” despite its limited efficacy. Given this limited efficacy, improved methods of treatment for PTCL are urgently required.

Andrei R. Shustov et al. (Blood (2016) 128 (22): 5355), which is hereby incorporated by reference in its entirety, discloses that Pralatrexate was the first drug approved in the US for the treatment of patients with relapsed or refractory PTCL (R/R PTCL). Pralatrexate is administered intravenously (IV) at a dose of 30 mg/m2 weekly for 6 weeks of a 7-week treatment cycle. Since pralatrexate and each of the components of the CHOP regimen target different aspects of tumor cell growth and proliferation, there is a potential for synergistic anti-tumor effect, and limited additive toxicities.

Andrei R. Shustov et al. (Blood (2017) 130 (Supplement 1): 818), which is hereby incorporated by reference in its entirety, discloses a Phase-I dose escalation study of Pralatrexate in combination with cyclophosphamide, doxorubicin, vincristine, and prednisone (CHOP). In Part 1 of this 3+3 dose-escalation study, pralatrexate was administered at 10, 15, 20, 25, or 30 mg/m2 as an IV push on days 1 and 8 of a standard 21-day CHOP regimen (cyclophosphamide 750 mg/m2, doxorubicin 50 mg/m2, vincristine 1.4 mg/m2 [maximum 2 mg] on day 1 and oral prednisone 100 mg on days 1-5). In Part 2 of the study patients were treated at the MTD of pralatrexate established in Part 1, with standard CHOP. In both parts of the study patients were treated with up to 6 cycles of therapy, or until toxicity or disease progression.

### SUMMARY OF THE INVENTION

The present disclosure provides compositions and methods for treating peripheral T-cell lymphoma featuring a combination of Pralatrexate, Cyclophosphamide, Vincristine, and Prednisone.

In accordance with the present disclosure, T cell non-Hodgkin's lymphoma is treated using a Pralatrexate and COP (Cyclophosphamide, Vincristine, and Prednisone) combination. Thus, in accordance with one aspect of the disclosure, a method is provided for the treatment of T cell non-Hodgkin's lymphoma comprising administering to a patient suffering from lymphoma a therapeutically effective amount of Pralatrexate and COP (Cyclophosphamide, Vincristine, and Prednisone) combination.

Methods for treating peripheral T-cell lymphoma (PTCL) are provided which may comprise administering to a subject in need thereof a combination comprising Cyclophosphamide, Vincristine, Prednisone and Pralatrexate,
wherein the combination does not comprise doxorubicin. In some embodiments, the combination consists of Cyclophosphamide, Vincristine, Prednisone and Pralatrexate. In various implementations, the administering occurs in a three-week cycle optionally comprising a drug holiday (e.g., a period of time during the cycle wherein no or different drugs are administered). In various implementations, the Cyclophosphamide, Vincristine, Prednisone and Pralatrexate are independently administered daily, weekly, or once every two weeks for at most two weeks of the three-week cycle, such that the three-week cycle contains a weeklong drug holiday. In some embodiments, the three-week cycle is repeated from at least 1 to 10 times. For example, the three-week cycle is repeated one time, two times, three times, four times, five times, six times, seven times, eight times, nine times, or ten times. In some embodiments, the Pralatextrate is administered on days 1 and 8 of a treatment cycle. In certain aspects, the Cyclophosphamide and/or Vinicristine, and/or Predinisone is administered daily for a time period within the treatment cycle. In some embodiments, the Cyclophosphamide and/or Vinicristine, and/or Predinisone is administered daily for days 1-15, or for days 1-10, or for days 1-8, or for days 1-6, or for days 1-5, or for days 1-4, or for days 1-3 of the treatment cycle.

The method may comprise a 21 day treatment cycle, the treatment cycle comprising:
a) administering Pralatrexate in a dosage of at least about 10 mg/m² intravenously on day 1 and day 8;
b) administering Cyclophosphamide in a dosage of at least about 700 mg/m² intravenously and Vincristine in a dosage of at least about 1 mg/m² intravenously, on day 1,
wherein the Cyclophosphamide and/or Vinicristine are independently optionally administered following or concomitantly with the administration of Pralatrexate on day 1; and
c) administering Prednisone in a dosage of at least about 50 mg orally on each of day 1 to day 5, wherein the prednisone is optionally administered on day 1 following the administration of Pralatrexate.

In certain embodiments, the 21 day cycle is repeated between 1 to 6 times. In certain aspects, the treatment cycle comprises at least one of:
a) administering Pralatrexate in a dosage of about 20 to about 30 mg/m² intravenously on day 1 and day 8;
b1) administering Cyclophosphamide in a dosage of from about 700 mg/m² to about 800 mg/m² on day 1;
b2) administering Cyclophosphamide in a dosage of about 750 mg/m² intravenously on day 1;
b3) administering Vincristine in a dosage of from about 1 mg/m² to about 2 mg/m² intravenously on day 1,
b4) administering Vinicristine in a dosage of about 1.2 mg/m² to about 1.6 mg/m² intravenously on day 1,
b5) administering Vinicristine in a dosage of about 1.4 mg/m², intravenously, on day 1,
c1) administering Prednisone in a dosage of from about 50 mg to 150 mg on day 1 following administration of Pralatrexate;
c2) administering Prednisone in a dosage of from about 80 mg to about 120 mg on day 1 following administration of Pralatrexate, and
c3) administering Prednisone in a dosage of about 100 mg orally on day 1 following administration of Pralatrexate.

Kits are also provided which may comprise Pralatrexate, Cyclophosphamide, Vincristine, and Prednisone, wherein said kit does not comprise doxorubicin. In some embodiments, the Pralatrexate, is formulated separately from the Cyclophosphamide, the Vincristine, and the Prednisone. In certain implementations, the kit may comprise a first pharmaceutical composition comprising about 20 to 30 mg/m² Pralatrexate and a first pharmaceutically acceptable excipient and/or a second pharmaceutical composition comprising about 500-1000 mg/m² Cyclophosphamide and a second pharmaceutically acceptable excipient, and/or a third pharmaceutical composition comprising about 1-3 mg/m² Vinicristine and a third pharmaceutically acceptable excipient, and/or a fourth pharmaceutical composition comprising about 50-150 mg Prednisone and a fourth pharmaceutically acceptable excipient. In certain aspects, at least two of the first pharmaceutical composition, second pharmaceutical composition, third pharmaceutical composition and fourth pharmaceutical composition are the same pharmaceutical composition. In some embodiments, the first pharmaceutical composition is an intravenous composition. In some embodiments, the second pharmaceutical composition is an intravenous composition. In various implementations, the third pharmaceutical composition is an intravenous composition. In some embodiments, the fourth pharmaceutical composition is an oral composition. In various implementations, the second composition comprises about 700-800 mg/m² Cyclophosphamide. In certain aspects, the second composition comprises about 750 mg/m² Cyclophosphamide. In some embodiments, the third composition comprises about 1-2 mg/m² Vinicristine. In certain implementations, the third composition comprises about 1.2-1.6 mg/m² Vinicristine. In some embodiments, the third composition comprises about 1.4 mg/m² Vincristine. In some embodiments, the fourth composition comprises about 75-125 mg Prednisone. In various aspects, the fourth composition comprises about 100 mg Prednisone.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991), each of which are hereby incorporated by reference in their entirety. As used herein, the following terms have the meanings ascribed to them below, unless specified otherwise.

By “ameliorate” is meant decrease, suppress, attenuate, diminish, arrest, or stabilize the development or progression of a disease.

By “combination therapy” is meant administration of two or more therapeutic agents in a coordinated fashion, and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “decreases” is meant a reduction by at least about 5% relative to a reference level. A decrease may be by 5%, 10%, 15%, 20%, 25% or 50%, or even by as much as 75%, 85%, 95% or more and any intervening percentages. The word decrease is not appearing in the text.

By “disease” is meant any condition or disorder that damages or interferes with the normal function of a cell, tissue, or organ. In some embodiments, the disease is T-cell lymphoma (e.g., lymphoblastic lymphomas, T cell prolymphocytic leukemia, T-cell granular lymphocytic leukemia, aggressive NK-cell leukemia, cutaneous T cell lymphoma (Mycosis fungoides/Sezary syndrome), anaplastic large cell lymphoma, T cell type, enteropathy-type T cell lymphoma, Adult T-cell leukemia/lymphoma including those associated with HTLV-1, and angioimmunoblastic T cell lymphoma, and subcutaneous panniculitic T cell lymphoma, peripheral T cell lymphomas).

By “effective amount” is meant the amount of a therapeutic agent required to ameliorate the symptoms of a disease relative to an untreated patient. Therapeutic agents described herein include Cyclophosphamide, Vincristine, Prednisone and Pralatrexate. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “reference” is meant a standard or control condition. In some embodiments, the effects of a combination therapy on a disease, such as PTCL, described herein are compared with the effects of a conventional therapy (e.g., CHOP) or on the effects of a disease in the absence of therapy.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

By “T-cell lymphoma” is meant a neoplasia characterized by the presence of cancerous T cell in a subject. T-cell lymphomas encompass a variety of conditions including without limitation:
(a) lymphoblastic lymphomas in which the malignancy occurs in primitive lymphoid progenitors from the thymus;
(b) mature or peripheral T cell neoplasms, including T cell prolymphocytic leukemia, T-cell granular lymphocytic leukemia, aggressive NK-cell leukemia, cutaneous T cell lymphoma (Mycosis fungoides/Sezary syndrome), anaplastic large cell lymphoma, T cell type, enteropathy-type T cell lymphoma, Adult T-cell leukemia/lymphoma including those associated with HTLV-1, and angioimmunoblastic T cell lymphoma, and subcutaneous panniculitic T cell lymphoma; and
(c) peripheral T cell lymphomas (PTCL) that initially involve a lymph node paracortex and never grow into a true follicular pattern.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Useful pharmaceutical carriers, excipients, and diluents for the preparation of the compositions hereof, can be solids, liquids, or gases. These include any and all solvents, dispersion media, coatings, antibacterial and antifungal agents, isotonic and absorption delaying agents and the like. The pharmaceutically acceptable carrier or excipient does not destroy the pharmacological activity of the disclosed compound and is nontoxic when administered in doses sufficient to deliver a therapeutic amount of the compound. Thus, the compositions can take the form of tablets, pills, capsules, suppositories, powders, enterically coated or other protected formulations (e.g., binding on ion-exchange resins or packaging in lipid-protein vesicles), sustained release formulations, solutions, suspensions, elixirs, and aerosols. The carrier can be selected from the various oils including those of petroleum, animal, vegetable or synthetic origin, e.g., peanut oil, soybean oil, mineral oil, and sesame oil. Water, saline, aqueous dextrose, and glycols are examples of liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, chitosan, talc, glucose, lactose, gelatin, malt, rice, flour, chalk, silica, magnesium stearate, sodium stearate, glycerol monostearate, sodium chloride, dried skim milk, glycerol, propylene glycol, water, and ethanol. The compositions may be subjected to conventional pharmaceutical additives such as preservatives, stabilizing agents, wetting or emulsifying agents, salts for adjusting osmotic pressure, and buffers. Suitable pharmaceutical carriers and their formulation are described in Remington's Pharmaceutical Sciences by E. W. Martin, which is hereby incorporated by reference in its entirety. Such compositions will, in any event, contain an effective amount of the active compound together with a suitable carrier so as to prepare the proper dosage form for administration to the recipient.

Non-limiting examples of pharmaceutically acceptable carriers and excipients include sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols, such as polyethylene glycol and propylene glycol; esters such as ethyl oleate and ethyl laurate; agar; buffering agents such as magnesium hydroxide and aluminum hydroxide; alginic acid; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening, flavoring and perfuming agents; preservatives; antioxidants; ion exchangers; alumina; aluminum stearate; lecithin; self-emulsifying drug delivery systems (SEDDS) such as d-atocopherol polyethyleneglycol 1000 succinate; surfactants used in pharmaceutical dosage forms such as Tweens or other similar polymeric delivery matrices; serum proteins such as human serum albumin; glycine; sorbic acid; potassium sorbate; partial glyceride mixtures of saturated vegetable fatty acids; water, salts or electrolytes such as protamine sulfate, disodium hydrogen phosphate, potassium hydrogen phosphate, sodium chloride, and zinc salts; colloidal silica; magnesium trisilicate; polyvinyl pyrrolidone; cellulose-based substances; polyacrylates; waxes; and polyethylene-polyoxypropylene-block polymers. Cyclodextrins such as α-, β-, and γ-cyclodextrin, or chemically modified derivatives such as hydroxyalkylcyclodextrins, including 2- and 3-hydroxypropyl-cyclodextrins, or other solubilized derivatives can also be used to enhance delivery of the compounds described herein.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DETAILED DESCRIPTION OF THE INVENTION

The present disclosure provides compositions and methods for treating peripheral T-cell lymphoma featuring a combination of Pralatrexate, Cyclophosphamide, Vincristine, and Prednisone.

Current methods for treating PTCL are inadequate. Aggressive combination chemotherapy regimens, such as hyper-CVAD (fractionated Cyclophosphamide, Vincristine, Adriamycin [doxorubicin], Dexamethasone, Methotrexate, Cytarabine) and VIP-ABVD (Vepesid [Etoposide], Ifosfamide, Platinum Agent [Cisplatin], alternating with Adriamycin [Doxorubicin], Bleomycin, Vinblastine, and Dacarbazine, exhibit toxicity and have not been shown to be superior to CHOP, which is the conventional therapy for PTCL.

### Pralatrexate

Pralatrexate is the first drug approved as a treatment for patients with relapsed or refractory peripheral T-cell lymphoma, or PTCL-a biologically diverse group of aggressive blood cancers. Pralatrexate is currently marketed under the trade name FOLOTYN®.

Pralatrexate is chemically known as “(2S)-24 [4-[(1RS)-1-[(2, 4-diaminopteridin-6-yl)methyl] but-3-ynyl] benzoyl]-amino]pentanedioic acid”, it is also known as “10-Propargy 1-10-deazaminopterin” or “PDX”. It has been tested and found useful in the treatment of cancer. In its racemic form, (2S)-24 [4-[(1RS)-1-[(2, 4-diaminopteridin-6-yl)methyl] but-3-ynylThenzoyl] amino]-pentanedioic acid has been approved by the U.S. Food and Drug Administration (FDA) as a treatment for relapsed and refractory peripheral T-cell lymphoma.

Pralatrexate was described in Journal of Medicinal Chemistry. 36:2228-2231 (1993) by DeGraw et al., and subsequently in U.S. Pat. Nos. 5,374,726, 5,354,741 and 6,028,071, each of which are hereby incorporated by reference in their entirety. It is represented by following structure:

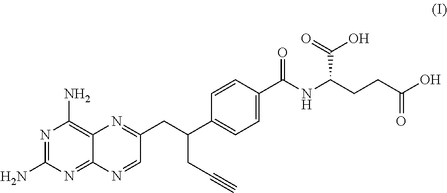 [SMILES: C#CCC(Cc1cnc2nc(N)nc(N)c2n1)c1ccc(C(=O)N[C@@H](CCC(=O)O)C(=O)O)cc1]

### CHOP Therapy

Cyclophosphamide also known as cytophosphane among other names, is a medication used as chemotherapy and to suppress the immune system. As chemotherapy it is used to treat lymphoma, multiple myeloma, leukemia, ovarian cancer, breast cancer, small cell lung cancer, neuroblastoma, and sarcoma. It is taken by mouth or injection into a vein.

Vincristine, also known as leurocristine and marketed under the brand name Oncovin among others, is a chemotherapy medication used to treat a number of types of cancer. This includes acute lymphocytic leukemia, acute myeloid leukemia, Hodgkin's disease, neuroblastoma, and small cell lung cancer among others. It is given intravenously.

Prednisone is a glucocorticoid medication mostly used to suppress the immune system and decrease inflammation in conditions such as asthma, COPD, and rheumatologic diseases. It is also used to treat high blood calcium due to cancer and adrenal insufficiency along with other steroids. It is taken orally.

Although CHOP/CHOP-like therapy is the currently recommended first-line treatment for PTCL, the prognosis remains poor, with most patients relapsing within 5 years. Therefore, improved treatment strategies are needed for patients with PTCL.

CHOP is generally considered as the preferred regimen in front line therapy of Non-Hodgkin's lymphoma. For the treatment of B-cell NHLs, immunotherapy drug Rituximab, is combined with chemotherapy combination and this combo is called R-CHOP. Further, A+CHP (Brentuximab Vedotin, Cyclophosphamide, Doxorubicin, and Prednisone) is used for the treatment of CD30-positive peripheral T-cell lymphomas (PTCL). For the lack of better alternative, CHOP has remained the standard of care for PTCL.

Although CHOP/CHOP-like therapy is the currently recommended first-line treatment for PTCL, the prognosis remains poor, with most patients relapsing within 5 years. Therefore, improved treatment strategies are needed for patients with PTCL.

Doxorubicin is often seen as a key component of CHOP, but has not been specifically evaluated in PTCL in single agent studies, as these studies were conducted in the 60s when PTCL was not identified as a distinct disease. However, a recent meta-analysis of the impact of Anthracycline-based regimens in PTCL concluded that the inclusion of Doxorubicin in a polychemotherapy regimen was not associated with a survival improvement. (AbouYabis A et al. ISRN Hematology Volume 2011, Article ID 623924, 14 pages).

With the goal of optimizing the development of a new front-line strategy, various approaches have been explored. These include moving away from multi-drug resistance (MDR)-related anthracycline-based regimens, such as standard CHOP, and incorporating novel agents (Pralatrexate) in up-front regimens.

Short-term toxicities of Doxorubicin and Pralatrexate are overlapping. Mucositis and hematologic toxicity are the main safety issues to manage in the clinic. To maintain the doses of CHOP as a backbone, in an embodiment, the present treatment regimen reduces Pralatrexate dose ever if the toxicity is related to CHOP drugs, in particular Doxorubicin. As such Pralatrexate would be systematically dose-reduced first.

Early clinical and subclinical cardiotoxicity was frequent in patients receiving a CHOP regimen. The threshold of the cumulative dose of doxorubicin appeared to be low: at doses >200 mg/m², 27% of patients had cardiac events. Elderly patients appeared to be at higher risk for such toxicity. The development of cardioprotective strategies or alternative treatments are urgently needed for aggressive Non-Hodgkin Lymphoma patients (S. Limat et. al. Annals of Oncology 14:277-281, 2003).

The inventors found that removal of doxorubicin from the CHOP treatment regimen results in a better safety and efficacy profile.

The present disclosure provides methods for treatment of peripheral T-cell lymphoma using a combination of Pralatrexate and COP.

The term COP refers to a triple combination of drugs, including Cyclophosphamide, Vincristine, and Prednisone.

### Treatment Regimen

The present disclosure further relates to methods for treatment of peripheral T-cell lymphoma (PTCL) using a combination of Pralatrexate and COP, for a treatment cycle of 21 days, wherein Pralatrexate is administered intravenously on Day 1 and Day 8; followed by administration of Cyclophosphamide on day 1; Vincristine IV on day 1; Prednisone 100 mg orally on day 1 (after the end of the Pralatrexate administration) to Day 5. The 21-day treatment cycle contains drug free period of at least 7 days and can be repeated for up to 10 cycles.

The present disclosure relates to methods for treatment of peripheral T-cell lymphoma (PTCL) using a combination of Pralatrexate and COP, wherein Pralatrexate 10 to 30 mg/m² is administered (e.g., intravenously) on Day 1 and Day 8; followed by administration of Cyclophosphamide 750 mg/m² is administered (e.g., intravenously) on day 1; Vincristine 1.4 mg/m² is administered (e.g., intravenously) on day 1; Prednisone 100 mg is administered (e.g., orally) on day 1 (after the end of the Pralatrexate administration) to Day 5, with cycles repeated every 21 days for up to 10 cycles

In some embodiments, Pralatrexate is administered intravenously on Day 1 and Day 8 as an IV push over 3 to 5 min, followed by and administration of Cyclophosphamide and Vincristine on Day 1 and Prednisone 100 mg for 5 days.

In some embodiments, Pralatrexate is administered intravenously on Day 1 and Day 8 as an IV push over 3 to 5 min. In some embodiments, the amount of Pralatrexate is 10, 20 or 30 mg/m².

In some embodiments, Prednisone 50 to 100 mg is administrated (e.g., orally) daily, wherein Day 1 (after the end of the Pralatrexate administration) for 5 days.

In another aspect, Cyclophosphamide 500 to 1000 mg/m² IV is administered on Day 1 after the end of the Pralatrexate administration.

In another aspect, Vincristine 1.2-1.6 mg/m² (maximum 2 mg) is administered intravenously on Day 1 after the end of the Pralatrexate administration.

In another aspect, the present disclosure relates to sequential administration of a combination of Pralatrexate+COP to the patients for the treatment of peripheral t-cell lymphoma (PTCL). Pralatrexate 20 or 30 mg/m² is administered intravenously on Day 1 and Day 8 as an IV push and COP is administered starting on Day 1 after the end of the Pralatrexate administration at a dose of Cyclophosphamide 750 mg/m² IV, Day 1; Vincristine 1.4 mg/m² (maximum 2 mg) IV, Day 1; Prednisone 100 mg orally (PO) daily; Day 1 (after the end of the Pralatrexate administration) to Day 5, with cycles repeated every 21 days for up to 10 cycles.

In another aspect Cyclophosphamide and/or Vinicristine, and/or Predinisone is administered daily for a time period within the treatment cycle.

In another aspect Cyclophosphamide and/or Vinicristine, and/or Predinisone is administered daily for days 1-15, or for days 1-10, or for days 1-8, or for days 1-6, or for days 1-5, or for days 1-4, or for days 1-3 of the treatment cycle.

In another aspect the 21 day treatment cycle comprises, a) administering Pralatrexate in a dosage of at least about 10 mg/m² intravenously on day 1 and day 8; b) administering Cyclophosphamide in a dosage of at least about 700 mg/m² intravenously and Vincristine in a dosage of at least about 1 mg/m² intravenously, on day 1, wherein the Cyclophosphamide and/or Vinicristine are independently optionally administered following or concomitantly with the administration of Pralatrexate on day 1; and c) administering Prednisone in a dosage of at least about 50 mg orally on each of day 1 to day 5, wherein the prednisone is optionally administered on day 1 following the administration of Pralatrexate.

Further aspect of the present invention relates to a kit comprising Pralatrexate, Cyclophosphamide, Vincristine, and Prednisone, wherein said kit does not comprise doxorubicin. Pralatrexate, in the kit is formulated separately from the Cyclophosphamide, the Vincristine, and the Prednisone.

In another aspect the kit comprises a first pharmaceutical composition comprising about 10 to 30 mg/m² Pralatrexate and a first pharmaceutically acceptable excipient and/or a second pharmaceutical composition comprising about 500-1000 mg/m² Cyclophosphamide and a second pharmaceutically acceptable excipient, and/or a third pharmaceutical composition comprising about 1-3 mg/m² Vinicristine and a third pharmaceutically acceptable excipient, and/or a fourth pharmaceutical composition comprising about 50-150 mg Prednisone and a fourth pharmaceutically acceptable excipient.

In another aspect of the kit, the first pharmaceutical composition is an intravenous composition, the second pharmaceutical composition is an intravenous composition, the third pharmaceutical composition is an intravenous composition and the fourth pharmaceutical composition is an oral composition.

In another aspect of the present invention the kit comprises about 700-800 mg/m² Cyclophosphamide, preferably 750 mg/m² of Cyclophosphamide, about 1-2 mg/m² of Vinicristine, preferably about 1.2-1.6 mg/m² of Vinicristine, most preferably about 1.4 mg/m² Vincristine, about 75-125 mg Prednisone, preferably about 100 mg Prednisone.

In accordance with the present disclosure, T cell non-Hodgkin's lymphoma is treated using Pralatrexate and COP (Cyclophosphamide, Vincristine, and Prednisone) combination. Thus, in accordance with one aspect of the disclosure, a method is provided for the treatment of T cell non-Hodgkin's lymphoma comprising administering to a patient suffering from lymphoma a therapeutically effective amount of Pralatrexate and COP (Cyclophosphamide, Vincristine, And Prednisone) combination.

The present disclosure provides a clinical trial protocol wherein the therapeutic effectiveness of Pralatrexate and COP combination is evaluated for the treatment of peripheral T-cell lymphoma. By removing Doxorubicin, the main CHOP component responsible for hematologic toxicity and mucositis, the compliance to Pralatrexate-COP would be greatly improved.

Certain specific aspects and embodiments of the invention is described in the following examples, which are provided solely for purposes of illustration and should not be construed to limit the scope of the invention in any manner.

The practice of the present invention employs, unless otherwise indicated, conventional techniques of molecular biology (including recombinant techniques),-microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991), each of which are hereby incorporated by reference in their entirety.

### Example-1

### 1. Dose Finding Study

This experiment was performed to identify the suitable dose of Pralatrexate that would be optimal in polychemotherapy for the next study (phase-III) based on safety and ORR (objective response rate) at 3 months. Initially, a lower dose of 20 mg/m² was compared to 30 mg/m² combined with COP. Therefore, these two dose levels were assigned randomly to 20 patients in addition to COP (Pralatrexate-COP). Appropriate dose for phase-III was determined based on safety and treatment compliance. If the two Pralatrexate-COP regimens are comparable, the regimen with the highest dose was selected. Dose effect on polyglutamate-Pralatrexate, DNA hypomethylation or H3-acetylation was evaluated in this part of the study as a surrogate for PD (pharmacodynamics) drug exposure. Determining the best regimen was based on several safety and compliance aspects. Selection of lower dose was done if treatment termination in more than 20% of the cases are related to safety. Further objective of dose finding study was also to evaluate the safety profiles for patients with newly diagnosed PTCL treated with Pralatrexate-COP compared to CHOP alone.

Dose finding study was a randomized Phase 2, open label, multicenter study in patients with PTCL who have not been previously treated and the control arm is CHOP, COP, (Cyclophosphamide, Vincristin, and Prednisone) in the literature, is the CHOP regimen without Doxorubicin (H). In the dose finding study, each investigational arm was having two dose levels which was compared with CHOP as reference. Treatment was randomized in three arms, Group 1a (Pralatrexate-COP) Pralatrexate 20 mg/m², Group 1b Pralatrexate 30 mg/m², all added to standard CHOP or COP respectively, and Group 2 for CHOP alone. Analysis was done when 100 patients have received at least one treatment cycle.

In new combination study, inventors found that Pralatrexate had a synergistic effect in combination with COP (Cyclophosphamide, Vincristine/Oncovin, And Prednisone), in patients with PTCL who have not been treated previously. Pralatrexate 20 or 30 mg/m² administered intravenously on Day 1 and Day 8 as an IV push over 3 to 5 min and COP is administered starting on Day 1 within 15 min (+5 min) after the end of the Pralatrexate administration at the doses, Cyclophosphamide 750 mg/m² IV, Day 1, Vincristine 1.4 mg/m² (maximum 2 mg) IV, Day 1, Prednisone 100 mg orally (PO) daily, Day 1 (after the end of the Pralatrexate administration) to Day 5 with cycles repeated every 21 days for up to 6 cycles. Combination therapy with Pralatrexate and COP may be used as a salvage front line therapy for PTCL in the newly diagnosed patients with a favorable safety profile.

### 2. Phase-III Clinical Trial

The objective of this phase-III clinical trial is to compare the progression-free survival (PFS) of patients with newly diagnosed peripheral T-cell lymphoma (PTCL) treated for up to 6 cycles with Pralatrexate in combination with COP (Pralatrexate-COP) to CHOP alone. It is a randomized, controlled, open-label, multicenter study in patients with PTCL who have not been treated previously. The optimal dose for Pralatrexate with COP determined after completion of dose finding study is evaluated in Phase-III clinical trial. In phase-III clinical trial each arm enrolls 150 patients and there are two treatment arms as described below. Patients with previously untreated PTCL are randomized in a balance manner (1:1) into 2 treatment groups and treated for up to 6 cycles.

### Group 1 (Pralatrexate-COP):

Pralatrexate 20 or 30 mg/m² is administered on Day 1 and Day 8 as an IV push over 3 to 5 min; COP is administered starting on Day 1 within 15 min (+5 min) after the end of the Pralatrexate administration at the doses, Cyclophosphamide 750 mg/m² IV, Day 1, Vincristine 1.4 mg/m² (maximum 2 mg) IV, Day 1, Prednisone 100 mg orally (PO) daily, Day 1 (after the end of the Pralatrexate administration) to Day 5 with cycles repeated every 21 days for up to 6 cycles.

### Group 2:

(CHOP): Combination chemotherapy to be administered starting on Day 1 at the doses shown below, with cycles repeated every 21 days for up to 6 cycles.
Cyclophosphamide 750 mg/m² IV, Day 1
Doxorubicin 50 mg/m² IV, Day 1 (limit lifetime cumulative dose to <550 mg/m² to reduce risk of cardiotoxicity)
Vincristine 1.4 mg/m² (maximum 2 mg) IV, Day 1
Prednisone 100 mg orally (PO) daily, Day 1 (after the end of the Pralatrexate administration for Groups 1) to Day 5.

### Example-2-Efficacy and Safety Study

NCT06072131, which is hereby incorporated by reference in its entirety, describes a Phase 3, randomized, open-label study comparing the efficacy and safety of the combination of BELEODAQ-CHOP OR FOLOTYN-COP to the CHOP regimen alone in newly diagnosed patients with Peripheral T-Cell Lymphoma. Briefly, the Arms, Interventions, and Treatments of the study are described in Table 1.

TABLE 1

| Arm | Intervention/Treatment |
|---|---|
| Active Comparator: Group 1a | Drug: Belinostat Injection |
| Group 1a Belinostat 600 mg/m2 + CHOP | Belinostat 600 mg/m2 or 1000 along with |
| | CHOP is givin in each cycle |
| | Other Name: Beleodaq ® |
| | Drug: CHOP |
| | CHOP is the comparator arm |
| | Other Name: Cyclophosphamide, |
| | Hydroxydaunorubicin (doxorubicin), |
| | Oncovin (vincristine), and Prednisone |
| Active Comparator: Group 1b | Drug: Belinostat Injection |
| Group 1b Belinostat 100 mg/m2 + CHOP | Belinosat 600 mg/m2 or 1000 mg/m2 along |
| | with CHOP is given in each cycle |
| | Other Name: Beleodaq ® |
| | Drug: CHOP |
| | CHOP is the comparator arm |
| | Other Name: Cyclophosphamide, |
| | Hydroxydaunorubicin (doxorubicin), |
| | Oncovin (vincristine), and Prednisone |
| Active Comparator: Group 2a | Drug: Pralatrexate Injection |
| Group 2a Pralatrexate 20 mg/m2 + COP | Pralatrexate 20 mg/m2 or 30 mg/m2 along |
| | with COP is given in each cycle |
| | Other Name: Folotyn ® |
| | Drug: CHOP |
| | CHOP is the comparator arm |
| | Other Name: Cyclophosphamide, |
| | Hydroxydaunorubicin (doxorubicin), |
| | Oncovin (vincristine), and Prednisone |
| Active Comparator: Group 2b | Drug: Pralatrexate Injection |
| | Pralatrexate 20 mg/m2 or 30 mg/m2 along |
| | with COP is given in each cycle |
| | Other Name: Folotyn ® |
| | Drug: |
| | COP is given in combination with |
| | Pralatrexate |
| | Other Name: Cyclophosphamide, Oncovin |
| | (vincristine), and Prednisone |
| Active Comparator: Group 3 | Drug: CHOP |
| CHOP | CHOP is the comparator arm |
| | Other Name: Cyclophosphamide, |
| | Hydroxydaunorubicin (doxorubicin), |
| | Oncovin (vincristine), and Prednisone |

Progression-free survival is determined from randomization to the first documented Progression of Disease or death, whichever occurs first.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for treating peripheral T-cell lymphoma (PTCL) comprising administering to a subject in need thereof a combination comprising Cyclophosphamide, Vincristine, Prednisone and Pralatrexate,
wherein the combination does not comprise doxorubicin.

2. The method according to claim 1, wherein the combination consists of Cyclophosphamide, Vincristine, Prednisone and Pralatrexate.

3. The method according to claim 1, wherein the administering occurs in a three-week cycle optionally comprising a drug holiday.

4. The method according to claim 3, wherein the Cyclophosphamide, Vincristine, Prednisone and Pralatrexate are independently administered daily, weekly, or once every two weeks for at most two weeks of the three-week cycle, such that the three-week cycle contains a weeklong drug holiday.

5. The method according to claim 3, wherein the three-week cycle is repeated from at least 1 to 10 times.

6. The method according to claim 5, wherein the three-week cycle is repeated one time, two times, three times, four times, five times, six times, seven times, eight times, nine times, or ten times.

7. The method according to claim 1, wherein the Pralatextrate is administered on days 1 and 8 of a treatment cycle.

8. The method according to claim 1, wherein the Cyclophosphamide and/or Vinicristine, and/or Predinisone is administered daily for a time period within the treatment cycle.

9. The method according to claim 8, wherein the Cyclophosphamide and/or Vinicristine, and/or Predinisone is administered daily for days 1-15, or for days 1-10, or for days 1-8, or for days 1-6, or for days 1-5, or for days 1-4, or for days 1-3 of the treatment cycle.

10. The method of claim 1, wherein the method comprises a 21 day treatment cycle, the treatment cycle comprising:
a) administering Pralatrexate in a dosage of at least about 10 mg/m² intravenously on day 1 and day 8;
b) administering Cyclophosphamide in a dosage of at least about 700 mg/m² intravenously and Vincristine in a dosage of at least about 1 mg/m² intravenously, on day 1,
wherein the Cyclophosphamide and/or Vinicristine are independently optionally administered following or concomitantly with the administration of Pralatrexate on day 1; and
c) administering Prednisone in a dosage of at least about 50 mg orally on each of day 1 to day 5, wherein the prednisone is optionally administered on day 1 following the administration of Pralatrexate.

11. The method of claim 10, wherein the 21 day cycle is repeated between 1 to 6 times.

12. The method of claim 10, wherein the treatment cycle comprises at least one of:
a) administering Pralatrexate in a dosage of about 20 to about 30 mg/m² intravenously on day 1 and day 8;
b1) administering Cyclophosphamide in a dosage of from about 700 mg/m² to about 800 mg/m² on day 1;
b2) administering Cyclophosphamide in a dosage of about 750 mg/m² intravenously on day 1;
b3) administering Vincristine in a dosage of from about 1 mg/m² to about 2 mg/m² intravenously on day 1,
b4) administering Vinicristine in a dosage of about 1.2 mg/m² to about 1.6 mg/m² intravenously on day 1,
b5) administering Vinicristine in a dosage of about 1.4 mg/m², intravenously, on day 1,
c1) administering Prednisone in a dosage of from about 50 mg to 150 mg on day 1 following administration of Pralatrexate;
c2) administering Prednisone in a dosage of from about 80 mg to about 120 mg on day 1 following administration of Pralatrexate, and
c3) administering Prednisone in a dosage of about 100 mg orally on day 1 following administration of Pralatrexate.

13. A kit comprising Pralatrexate, Cyclophosphamide, Vincristine, and Prednisone, wherein said kit does not comprise doxorubicin.

14. The kit of claim 13, wherein the Pralatrexate, is formulated separately from the Cyclophosphamide, the Vincristine, and the Prednisone.

15. The kit of claim 13, wherein the kit comprises a first pharmaceutical composition comprising about 20 to 30 mg/m² Pralatrexate and a first pharmaceutically acceptable excipient and/or a second pharmaceutical composition comprising about 500-1000 mg/m² Cyclophosphamide and a second pharmaceutically acceptable excipient, and/or a third pharmaceutical composition comprising about 1-3 mg/m² Vinicristine and a third pharmaceutically acceptable excipient, and/or a fourth pharmaceutical composition comprising about 50-150 mg Prednisone and a fourth pharmaceutically acceptable excipient.

16. The kit according to claim 15, wherein at least two of the first pharmaceutical composition, second pharmaceutical composition, third pharmaceutical composition and fourth pharmaceutical composition are the same pharmaceutical composition.

17. The kit of claim 15, wherein the first pharmaceutical composition is an intravenous composition, the second pharmaceutical composition is an intravenous composition, the third pharmaceutical composition is an intravenous composition, the fourth pharmaceutical composition is an oral composition, or a combination thereof.

18-20. (canceled)

21. The kit of claim 15, wherein the second composition comprises about 700-800 mg/m² Cyclophosphamide, the third composition comprises about 1-2 mg/m² Vinicristine, the fourth composition comprises about 75-125 mg Prednisone, or a combination thereof.

22. The kit of claim 15, wherein the second composition comprises about 750 mg/m² Cyclophosphamide, the third composition comprises about 1.4 mg/m² Vinicristine, the fourth composition comprises about 100 mg Prednisone, or a combination thereof.

23. (canceled)

24. The kit of claim 15, wherein the third composition comprises about 1.2-1.6 mg/m² Vinicristine.

25-27. (canceled)
